(19) United States
(12) Patent Application Publication
(10) Pub. No.: US 2018/0217156 A1
(43) Pub. Date: Aug. 2, 2018

(54) MEANS AND METHODS FOR A SAMPLE PREPARATION, ESPECIALLY FOR MASS SPECTROMETRY

(71) Applicant: MAX-PLANCK-GESELLSCHAFT ZUR FORDERUNG DER WISSENSCHAFTEN E.V., München (DE)

(72) Inventors: Nils A. KULAK, München (DE); Garwin PICHLER, München (DE); Matthias MANN, Stockdorf (DE)

(21) Appl. No.: 15/747,431

(22) PCT Filed: Jul. 28, 2016
(86) PCT No.: PCT/EP2016/068095

§ 371 Date: Jan. 24, 2018

(30) Foreign Application Priority Data
Jul. 29, 2015 (EP) 15178894.0

(51) Int. Cl. G01N 33/68

(52) U.S. Cl. CPC G01N 33/6848; G01N 2560/00; G01N 2458/15

(57) ABSTRACT

The present invention relates to a use of a tertiary amine as buffer in sample preparation, preferably for mass spectrometry and/or UV/vis spectroscopy, wherein the sample comprises proteins, polypeptides and/or peptides, and said sample preparation comprises: (a) protein, polypeptide and peptide denaturation; and (b) chemical isotope labelling and/or chemical cross-linking, wherein said sample preparation does not use primary amine buffers.

### RELATED APPLICATIONS

This application is the U.S. National Phase of International Application No. PCT/EP2016/068095, filed Jul. 28, 2016, which claims priority to European Patent Application No. 15178894.0, filed Jul. 29, 2015. The subject matter of each of these applications is incorporated herein by reference in their entirety.

The present invention relates to a use of a tertiary amine as buffer in sample preparation, preferably for mass spectrometry and/or UV/vis spectroscopy, wherein the sample comprises proteins, polypeptides and/or peptides, and said sample preparation comprises: (a) protein, polypeptide and peptide denaturation; and (b) chemical isotope labelling and/or chemical cross-linking, wherein said sample preparation does not use primary amine buffers.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Bottom-up proteomics platforms are quickly advancing in performance and throughput. Mass spectrometers are improving in acquisition speed and sensitivity to achieve proteomic depth in shorter time. Sample preparation is a very important component of the overall workflow and remains to be a limiting factor for high-throughput MS-based proteomics. Throughput can be improved by sample multiplexing using isotope labelling technologies which modify the mass of the analytes. The analytes are then almost identical in their biochemical behavior but differ in their mass which can be observed in a mass spectrometer. Therefore, differently isotope labeled samples can be mixed prior to LC-MS analysis which leads to a higher complexity of the sample but also a higher overall throughput (Ong, S. E. and M. Mann, Mass spectrometry-based proteomics turns quantitative. Nat Chem Biol, 2005. 1(5): p. 252-62).

Isotope labelling technologies include stable isotope labelling by amino acids in cell culture (SILAC) (Ong, S. E., et al., Stable isotope labelling by amino acids in cell culture, SILAC, as a sample and accurate approach to expression proteomics. Mol Cell Proteomics, 2002. 1(5): p. 376-86) and chemical derivatization such as isotope-coded affinity tags (ICAT) (Yi, E. C., et al., Increased quantitative proteome coverage with (13)C/(12)C-based, acid-cleavable isotope-coded affinity tag reagent and modified data acquisition scheme. Proteomics, 2005. 5(2):p. 380-7) and isobaric mass tags (TMT, iTRAQ) (Thompson, A., et al., Tandem mass tags: a novel quantification strategy for comparative analysis of complex protein mixtures by MS/MS. Anal Chem, 2003. 75(8): p. 1895-904; Ross, P. L., et al., Multiplexed protein quantitation in Saccharomyces cerevisiae using amine-reactive isobaric tagging reagents. Mol Cell Proteomics, 2004. 3(12): p. 1154-69). Chemical labelling is known for its multiplexing capability and versatility of sample labelling options. Most chemical labelling reagents are based on N-hydroxy succinimide (NHS) ester reactive groups which react and covalently modify primary amine groups. However, side products and chemical background, especially primary amines may quench labelling efficiency and thereby decrease measurement performance. Manufacturers of such labelling reagents recommend enriching proteins and removing any chemicals prior to labelling, typically by precipitation in order to allow efficient reaction with the NHS ester label.

The typical workflow used in sample preparation prior to chromatography and MS analysis involves the following steps. Cellular and tissue material is subjected to lysis. Prior to lysis, cross-linking such as chemical cross-linking may be performed. Cross-linking may be a means of elucidating in vivo interactions. Typical agents for cross-linking are NHS esters. Subsequent to lysis, denaturation is performed. Disulfide bridges are reduced, and the resulting sulfhydryl groups are alkylated. The resulting material is subjected to proteolysis, in many instances with trypsin. Subsequent thereto, chemical isotope labelling may be performed. Given that reagents used for the preceding steps interfere negatively with the chemical labelling process, the labelling step has to be preceded by a cumbersome purification step. This purification step involves precipitation, centrifugation and re-suspension. Not only are these steps cumbersome, laborious and require hand-on time, but furthermore they typically require at least 100 μg of protein; otherwise there is significant risk of sample loss and even complete sample loss.

In view of the deficiencies of the prior art, the technical problem underlying the present invention can be seen in the provision of alternative or improved means and methods for sample preparation, especially for mass spectrometry or UV/vis spectroscopy.

In a first aspect, the present invention provides an alkylating agent comprising or consisting of an N,N-dialkyl haloalkanamide, wherein (i) each alkyl is independently chosen from C₁ to C₅ unbranched or branched alkyl; (ii) alkane is unbranched or branched C₂ to C₅ alkane; and (iii) halogen is chosen from chlorine, bromine and iodine, wherein each of said alkyl and said haloalkanamide may independently be substituted, substituents including OH.

The term “alkylating agent” designates a composition of matter which is capable of introducing alkyl moieties in a target molecule. Typically, alkylation occurs at reactive groups of the target molecule. A preferred reactive group in accordance with the present invention is the sulfhydryl group. A preferred type of target molecule in accordance with the present invention are proteins, polypeptides and peptides.

The designation of the alkylating agent as an haloalkanamide follows standard nomenclature. To explain further, an alkanamide is an alkane wherein the carbon at position number 1 is an amide. For example, ethanamide is also known in the art as acetamide. In fact, acetamide is a preferred alkanamide in accordance with the present invention.

The alkylating agents disclosed above are alkanamide which carry at least three substituents: two alkyl moieties bound to the amide nitrogen, and a halogen in the alkane moiety of the alkanamide.

While not being preferred, it is envisaged that further heteroatoms are present; examples include hydroxyl groups which may be attached to any one of the alkyl moieties and/or to the alkane. Preference is given to one hydroxyl group per alkane or alkyl moiety.

The terms “unbranched” and “branched” have their art-established meaning. Unbranched alkyl or alkane moieties, respectively, are also known as n-alkyl and n-alkanes. On the other hand, isopropyl is an example of a branched alkyl moiety. 2-methyl propanamide is an example of a branched C₄ alkanamide.

In a second aspect, the present invention provides the use of an N,N-dialkyl haloalkanamide as defined in the first aspect for alkylating.

The following preferred embodiments relate to both the alkylating agent in accordance with the first aspect and the use in accordance with the second aspect of the invention.

In a preferred embodiment, both occurrences of alkyl are methyl or ethyl. This embodiment relates to N,N-dimethyl haloalkanamides as well as N,N-diethyl haloalkanamides.

In a further preferred embodiment, the alkane is ethane. In other words, the alkanamide is an acetamide.

In further preferred embodiments of any of the above aspects as well as preferred embodiments, (a) said halogen is chlorine; and/or (b) said halogen is at position 2 of said haloalkanamide.

In other words, and even though preference is given to acetamides, the present invention extends to, for example, 2-chloro-propanamides with alkyl substituents as defined above on the nitrogen. Accordingly, the present invention also provides an alkylating agent comprising or consisting of an N,N-dialkyl haloalkanamide, wherein (i) each alkyl is independently chosen from C₃ to C₅ unbranched or branched alkyl; (ii) alkane is unbranched or branched C₃ to C₅ alkane; and (iii) halogen is chosen from chlorine, bromine and iodine, wherein each of said alkyl and said haloalkanamide may independently be substituted, substituents including OH; and wherein said halogen is at position 2 of said haloalkanamide.

As noted above, preference is given to none of said alkyl moieties being substituted, and furthermore to the haloalkanamide not carrying any further substituents other than halogen.

In an especially preferred embodiment, said agent is 2-Chloro-N,N-dimethylacetamide or 2-Chloro-N,N-diethylacetamide.

In a further preferred embodiment, said alkylating is alkylating of proteins, polypeptides and/or peptides, preferably of —SH groups in said proteins, polypeptides and/or peptides.

In the prior art, for the purpose of cysteine alkylation, use is made routinely of 2-iodoacetamide (IAA) or 2-chloroacetamide (CAA). Such alkylating agents, however, are capable of reacting with chemical labelling agents such as NHS esters. Therefore, the presence of the art-established alkylating agents in the reaction mix for chemical labelling (the same applies for chemical cross-linking) is undesirable because the intended alkylation reaction (or cross-linking reaction) would be quenched owing to the presence of IAA or CAA. The present inventors surprisingly discovered that the use of alkylating agents which are tertiary amines is a means of circumventing this problem. Using the novel alkylating agents according to the present invention helps to render the above described cumbersome step of precipitation dispensable. In fact, precipitation—an indispensable step in the art-established procedures—is performed inter alia in order to get rid of the alkylating agents which have been used in the alkylating step preceding the chemical labelling step.

A further advantage of the novel alkylating agents in accordance with the present invention is that, in contrast to the art-established alkylating agents, they are not solid but liquid. Accordingly, there is no need to weigh the alkylating agents; instead they can be pipetted.

The terms “protein”, “polypeptide” and “peptide” have their art-established meanings. Peptides and polypeptides are single molecules, wherein proteins may be of dimeric, oligomeric or multimeric structure. Proteins may be associated with non-proteinaceous molecules, wherein such association may be covalent or non-covalent. The monomeric units in dimeric, oligomeric or multimeric proteins are polypeptides. Either one of peptides and polypeptides is a polycondensate of amino acids, wherein peptides consists of up to and including 30 amino acids, and polypeptides of more than 30 amino acids.

The monomeric building blocks of peptides, polypeptides and proteins are preferably the 20 standard α-amino acids. Having said that, other naturally occurring or non-naturally occurring amino acids are deliberately envisaged as building block. Examples thereof are selenomethonine, pyrrolysine and hydroyproline. Ornitine and canavanine are further atypical amino acids.

Peptides and polypeptides may contain post-translational modifications such as phosphorylation, glycation, glycosylation and methylation. These and other post-translational modifications are well-known in the art as are the typical attachment sites in peptides, polypeptides and proteins.

In a third aspect, the present invention provides a method of sample preparation, preferably for mass spectrometry and/or UV/vis spectroscopy, the sample comprising proteins, polypeptides and/or peptides, said method comprising or consisting of the step of alkylation of said proteins, polypeptides and/or peptides with the agent defined above.

The novel alkylating agents of the invention can conveniently be used for any type of sample preparation. Especially the type of sample preparation needed for mass spectrometry and UV/vis spectroscopy benefits significantly from the simplified workflow rendered possible by said alkylating agents.

The above disclosed aspects relate to a specific improvement during sample preparation, especially for mass spectroscopy as well as any other spectroscopic applications which aim at detecting chemically labeled, such as chemically isotope labeled and/or chemically cross-linked molecular species in a sample. The improvement is the use of better alkylating agents. The present inventors, aiming at the consistent avoidance of agents which may negatively interfere with chemical isotope labelling or chemical cross-linking, provided a further substantial improvement. In the prior art sample preparation or at least certain steps thereof are effected in buffers which contain primary amines such as Tris buffer. This is a second source of primary amines. Primary amines, however, quench the reaction of chemical labelling agents, especially NHS esters with their respective target molecules. In order to render the above-described step of precipitation and re-suspension fully dispensable, the present inventors proceeded further to implement a procedure which deliberately avoids primary amine buffers.

Accordingly, the present invention, in a fourth aspect, provides use of a tertiary amine as buffer in sample preparation, preferably for mass spectrometry (MS) and/or UV/vis spectroscopy, wherein the sample comprises proteins, polypeptides and/or peptides, and said sample preparation comprises: (a) protein, polypeptide and peptide denaturation; and (b) chemical isotope labelling and/or chemical cross-linking.

As explained above, protein and polypeptide denaturation is the first compulsory step in sample preparation. The whole sample preparation workflow is described above and is furthermore the subject of the present invention as detailed further below.

Generally speaking, the invention provides means and methods for simplifying sample preparation for spectroscopic methods, in particular mass spectrometry. Said simplifying is rendered possible by the consistent avoidance of agents which comprise primary amine groups. As noted above, primary amines quench the reaction of sample constituents, in particular proteins and peptides with chemical labeling agents and cross-linking agents. Secondary amines are less critical in that respect. In a particularly preferred embodiment, agents comprising secondary amine groups are avoided as well.

The prior art methods typically use primary amine agents such as buffers comprising primary amine groups. In order to avoid the mentioned quenching, the prior art processes typically involve a separation step such as precipitation. In the following, an exemplary comparison is provided between prior art, sample preparation and sample preparation in accordance with the present invention.

Prior art sample preparation for MS typically comprises: (1) solubilizing, reducing and alkylating proteins in buffers which comprise primary amine groups, (2) precipitation of the proteins in order to remove said buffers as well as salts, for example by adding acetone, (3) re-suspension of the proteins by using buffers which do not comprise primary amine groups, (4) proteolytic digestion of the proteins to yield peptides, and (5) labelling of primary amine groups of the peptides by using agents which are activated with an NHS ester. According to the invention, steps (2) and (3) are dispensable, i.e. subsequent to step (1), proteolytic digestion and labelling can be performed without any intervening steps, i.e. in the same buffer and preferably in the same vessel.

The avoidance of primary amine agents and the consistent use of tertiary amine agents, be it tertiary amine buffers and/or tertiary amine alkylation agents, enables a simpler workflow. Said simpler workflow is characterized by the absence of precipitation, in particular protein precipitation.

In a preferred embodiment of the use according to the fourth aspect, said tertiary amine is selected from (a) trimethylammonium salts, preferably trimethylammonium bicarbonate (TEAB), trimethylammonium formate (TEAF) and trimethylammonium acetate (TEAA); and (b) zwitter-ionic buffer substances comprising one or more nitrogens, said one or more nitrogens being tertiary amine nitrogens, said zwitter-ionic buffer substances preferably being selected from HEPES, MOPS, HEPPS and MES.

These buffers abstances share the feature of being tertiary amines. While being used for certain procedures of the prior art, the present inventors for the first time recognized the distinct advantage of using these buffers throughout the entire process of sample preparation. In fact, their use provides for avoiding primary amines which previously have been introduced via buffer abstances. As such, protein precipitation, which is very difficult to implement in robotic systems and therefore in practical for high throughput sample handling, becomes fully dispensable. Avoiding protein precipitation allows to further automate the chemical labelling and thereby improve reproducibility and robustness of the procedure. While in the prior art typically at least 100 μg of protein were necessary to robustly precipitate and wash the protein pellet, the labelling methods enabled by the present invention and described in more detail below allow much higher sensitivity, may allow single molecule labelling, and are highly suitable for common protein quantities analyzed in LC-MS experiments, ranging from about 2 to about 20 μg of protein.

In a further preferred embodiment of the use according to the first aspect, said sample preparation comprises or consists of (a) optionally chemical cross-linking; (b) optionally cell lysis; (c) protein, polypeptide and peptide denaturation; (d) reduction; (e) alkylation; (f) proteolysis; and (g) optionally chemical isotope labelling, provided that at least one of chemical cross-linking according to (a) and chemical isotope labelling according to (g) is performed.

Chemical cross-linking and chemical isotope labelling are two steps which typically make use of agents that are quenched by primary amines. The preferred pH range for performing chemical isotope labelling as well as chemical cross-linking is between 7 and 9. Outside this interval, side reactions may occur.

In a fifth aspect, the present invention provides a method of sample preparation, preferably for mass spectrometry and/or UV/vis spectroscopy, the sample comprising proteins and/or polypeptides and/or peptides, said method comprising or consisting of the following steps, wherein said steps are performed in the same buffer, said buffer being a tertiary amine, preferably a triethylammonium salt or a zwitter-ionic buffer substance as defined above, said method comprising or consisting of: (a) optionally chemical cross-linking; (b) optionally cell lysis, wherein steps (a) and (b) may be performed in any order; (c) protein, polypeptide and peptide denaturation; (d) reduction; (e) alkylation; (f) proteolysis; and (g) optionally chemical isotope labelling, provided that at least one of chemical cross-linking according to (a) and chemical isotope labelling according to (g) is performed.

One or more intervening purification steps may be done after one or more of steps (a), (b), (e), (f) and/or (g). Preferred is performing purification only after step (g), to the extent step (g) is actually performed. A preferred method of purification is disclosed in European patent application 15 17 6142.6.

In a preferred embodiment of the use in accordance with the fourth and of the method in accordance with the fifth aspect, said alkylation according to (d) is effected with the agent as defined in accordance with the first aspect.

These preferred uses and methods are characterized in that (i) they employ a tertiary amine buffer for the entire use or method, respectively, (ii) they do not employ agents which comprise primary amine groups, (iii) for the purpose of alkylation, agents are used which are tertiary amines, and, consistent with (ii), (iv) no alkylation reagents are used which comprise primary amine groups.

In a further preferred embodiment of said use and said method, the sample preparation does not involve precipitation. As explained above, it is the inventors' contribution to establish a sample preparation protocol which renders precipitation fully dispensable. While precipitation could still be performed, this is clearly not the intention and therefore not preferred.

In a sixth aspect, the present invention provides a kit comprising or consisting of (a) an agent as defined in accordance with the first aspect; (b) a buffer which is tertiary amine, said tertiary amine preferably being a triethylammonium salt or a zwitter-ionic buffer substance as defined above.

In a preferred embodiment, said kit may further comprise one or more of the following: (c) a reducing agent, preferably TCEP or DTT; (d) a proteolytic enzyme, preferably trypsin; (e) one or more isotope labelling agents, preferably NHS esters; (f) one or more cross-linking agents, preferably NHS esters; and (g) a manual containing instructions for performing the method of the fifth aspect of the present invention.

Tris(2-carboxyethyl)phosphine (TCEP) and dithiothreitol (DTT) are both art-established reducing agents. They are suitable for reducing also in accordance with the improved sample preparation workflow in accordance with the present invention.

The use of proteolytic enzymes in the course of sample preparation for mass spectrometry is art-established. It renders fragments which are convenient to handle and analyze. The choice of the proteolytic enzyme is not particularly limited; any of the art-established enzymes may be used, wherein preference is given to trypsin.

As noted above, both chemical isotope labelling as well as chemical cross-linking share the feature that preferred agents for either procedure are N-hydroxysuccinimide (NHS) esters. NHS esters of carboxylic acids are also known as activated carboxylic acids, given that the ester between the carboxylic acid and NHS is semi-stable. The use of NHS esters for labeling as such and in the field of mass spectrometry is art-established; see, e.g. Quantitative Methods in Proteomics, Methods in Molecular Biology, Volume 893, 2012, pp 85-100 and Rappsilber J. (2011), The beginning of a beautiful friendship: Cross-linking/mass spectrometry and modeling of proteins and multi-protein complexes, J. Struct. Biol. 173(3):530-540.

Preferred labelling reagents are TMT (Tandem Mass Tag), iTRAQ (Isobaric tags for relative and absolute quantitation), mTRAQ; and preferred cross-linker are DSSO (disuccinimidyl sulfoxide) and DSS (Disuccinimidyl suberate).

In a seventh aspect, the present invention provides the use of the kit of the sixth aspect for alkylating proteins, polypeptides or peptides. In a preferred embodiment of said use, said use is furthermore for chemical isotope labelling.

1. Use of a tertiary amine as buffer in sample preparation, preferably for mass spectrometry and/or UV/vis spectroscopy, wherein the sample comprises proteins, polypeptides and/or peptides, and said sample preparation comprises:
(a) protein, polypeptide and peptide denaturation; and
(b) chemical isotope labelling and/or chemical cross-linking,
wherein said use does not involve primary amine buffers.

2. The use of claim 1, wherein said tertiary amine is selected from
(a) trimethylammonium salts, preferably trimethylammonium bicarbonate (TEAB), trimethylammonium formate (TEAF) and trimethylammonium acetate (TEAA); and
(b) zwitter-ionic buffer substances comprising one or more nitrogens, said one or more nitrogens being tertiary amine nitrogens, said zwitter-ionic buffer substances preferably being selected from HEPES, MOPS, HEPPS and MES.

3. The use of claim 1 or 2, wherein said sample preparation comprises or consists of
(a) optionally chemical cross-linking;
(b) optionally cell lysis;
(c) protein, polypeptide and peptide denaturation;
(d) reduction;
(e) alkylation;
(f) proteolysis; and
(g) optionally chemical isotope labelling,
provided that at least one of chemical cross-linking according to (a) and chemical isotope labelling according to (g) is performed.

4. A method of sample preparation for mass spectrometry and/or UV/vis spectroscopy, the sample comprising proteins, polypeptides and/or peptides, said method comprising or consisting of the step of alkylation of said proteins, polypeptides and/or peptides with an alkylating agent comprising or consisting of an N,N-dialkyl haloalkanamide, wherein
(i) each alkyl is independently chosen from C₁ to C₅ unbranched or branched alkyl;
(ii) alkane is unbranched or branched C₂ to C₅ alkane; and
(iii) halogen is chosen from chlorine, bromine and iodine,
wherein each of said alkyl and said haloalkanamide may independently be substituted, substituents including OH.

5. A method of sample preparation for mass spectrometry and/or UV/vis spectroscopy, the sample comprising proteins, polypeptides and/or peptides, said method comprising or consisting of the following steps, wherein said steps are performed in the same buffer, said buffer being a tertiary amine, preferably as defined in claim 2:
(a) optionally chemical cross-linking;
(b) optionally cell lysis, wherein steps (a) and (b) may be performed in any order;
(c) protein, polypeptide and peptide denaturation;
(d) reduction;
(e) alkylation;
(f) proteolysis; and
(g) optionally chemical isotope labelling,
provided that at least one of chemical cross-linking according to (a) and chemical isotope labelling according to (g) is performed,
wherein said method does not use primary amine buffers.

6. The use of claim 3 or the method of claim 5, wherein said alkylation according to (e) is effected with an alkylating agent comprising or consisting of an N,N-dialkyl haloalkanamide, wherein
(i) each alkyl is independently chosen from C₁ to C₅ unbranched or branched alkyl;
(ii) alkane is unbranched or branched C₂ to C₅ alkane; and
(iii) halogen is chosen from chlorine, bromine and iodine,
wherein each of said alkyl and said haloalkanamide may independently be substituted, substituents including OH.

7. The use of any one of claim 1 to 3 or 6, or the method of any one of claims 4 to 6, wherein said sample preparation does not involve precipitation, preferably protein precipitation.

8. A kit comprising or consisting of:
(a) an alkylating agent comprising or consisting of an N,N-dialkyl haloalkanamide, wherein
(i) each alkyl is independently chosen from C₁ to C₅ unbranched or branched alkyl;
(ii) alkane is unbranched or branched C₂ to C₅ alkane; and
(iii) halogen is chosen from chlorine, bromine and iodine,
wherein each of said alkyl and said haloalkanamide may independently be substituted, substituents including OH;
(b) a buffer which is a tertiary amine, said tertiary amine preferably being as defined in claim 2.

9. Use of the kit of claim 8 for alkylating proteins, polypeptides or peptides, and preferably furthermore for chemical isotope labelling.

10. The method, use or kit of any one of claim 4, 6 or 8, respectively, wherein both occurrences of alkyl in said agent are methyl or ethyl.

11. The method, use or kit of any one of claim 4, 6, 8 or 10, wherein the alkane in said agent is ethane.

12. The method, use or kit of any one of claim 4, 6, 8, 10 or 11, wherein in said agent
(a) said halogen is chlorine; and/or
(b) said halogen is at position 2 of said haloalkanamide.

13. The method, use or kit of any one of claims 4, 6, 8 or 10 to 12, wherein neither of said alkyl is substituted and said haloalkanamide is not substituted.

14. The method, use or kit of any one of claims 4, 6, 8 or 10 to 13, wherein said agent is 2-Chloro-N,N-dimethylacetamide or 2-Chloro-N,N-diethylacetamide.

15. An alkylating agent comprising or consisting of an N,N-dialkyl haloalkanamide, wherein
(i) each alkyl is independently chosen from C₃ to C₅ unbranched or branched alkyl;
(ii) alkane is unbranched or branched C₃ to C₅ alkane; and
(iii) halogen is chosen from chlorine, bromine and iodine,
wherein each of said alkyl and said haloalkanamide may independently be substituted, substituents including OH;
and wherein said halogen is at position 2 of said haloalkanamide.
